(19) United States
(12) Patent Application Publication
(10) Pub. No.: US 2015/0374729 A1
(43) Pub. Date: Dec. 31, 2015

(54) DISINFECTANT DELIVERY SYSTEM AND METHOD FOR DISINFECTION

(71) Applicant: THE CLOROX COMPANY, Oakland, CA (US)

(72) Inventors: John Glauber, Pleasanton, CA (US); Matthew Harrington, Pleasanton, CA (US); Casper W. Chiang, Danville, CA (US)

(73) Assignee: THE CLOROX COMPANY, Oakland, CA (US)

(21) Appl. No.: 14/315,005

(22) Filed: Jun. 25, 2014

(51) Int. Cl. A61K 31/7004; A61K 31/045; A01N 25/08; A61B 19/02; A01N 37/02; A01N 31/02; A61J 1/05; A61K 31/191; A01N 47/44

(52) U.S. Cl. CPC A61K 31/7004; A61K 31/191; A61K 31/045; A01N 25/08; A01N 47/44; A01N 37/02; A01N 31/02; A61J 1/05; A61B 19/0271

(57) ABSTRACT

The invention relates to methods and kits for use in reducing microbial counts on a patient's skin prior to surgery. Such a kit may be sealed, and include a dry substrate, a container of antiseptic composition, a tray with recessed portion(s), and a cover in sealing contact with a top rim of the tray to seal the contents in the tray. Such a kit may include two (e.g., identical) trays, which are initially attached to one another, each tray providing contents sufficient for one use (e.g., one use within 2 hours of the scheduled surgery, and a prior use done earlier (e.g., the night before). The related method may include providing the kit, removing the cover, dosing the substrate with the antiseptic composition in the tray's recessed region, and using the dosed substrate to apply the composition to skin proximate a location of the invasive procedure to be performed.

### BACKGROUND OF THE INVENTION

1. The Field of the Invention

The present invention is directed to kits and related methods for reducing microbial counts on a patient's skin prior to an invasive procedure (e.g., surgery), for example, to reduce risk of post-operative infection.

2. Description of Related Art

Antiseptic preparation of patient's skin immediately prior to surgery conventionally includes a two to ten minute scrubbing of the affected area with an antiseptic soap solution followed by the application of a water-soluble antiseptic composition. In some cases, it may be beneficial to provide the patient with a cleansing kit that may be used by the patient himself or herself, at home. HIBICLENS, 4% chlorhexidine gluconate (CHG) solution and CareFusion's SMARTCLEAN cleansing kit and CHG solution are examples of such kits. The solutions that are currently available on the market maybe sold as separate items: antiseptic solution, cloths/sponges, tray, etc. Alternatively, these items may be sold as a kit packaged in a non-sterile pouch or other suitable packaging, but none of the currently available kits come in a sterilized tray that is ready for use. In addition, none of the kits currently available provide a solution to having multiple uses where the kit may be separated into two more components while preserving sterility. This feature allows portions of the kit to be used multiple times in different locations while maintaining a sterile kit for each use.

In view of the current state of the art, there is a continuing need for improved kits and methods that may be employed to reduce risk of infection by reducing microbial counts on a patient's skin, particularly kits that are configured to provide improved ease of use to the patient at home, prior to the invasive procedure.

### BRIEF SUMMARY

In an embodiment, the present invention is directed to methods and related kits for reducing microbial count on a patient's skin prior to an invasive procedure. Such kits and methods may aid in reducing risk of post-operative or other infection. According to an embodiment, the method includes providing a sealed kit comprising one or more dry substrates, a container containing an antiseptic composition, a tray with one or more recessed portions for holding the dry substrates and the antiseptic composition, and a cover in contact with a top rim of the tray, which cover seals the kit. The method further comprises removing the cover from the tray, dosing the dry substrate with the antiseptic composition within the recessed portion of the tray, and using the dosed substrate to apply the antiseptic composition to at least one area of skin proximate a location of the invasive procedure to be performed.

Such methods may reduce microbial (e.g., bacterial, viral, fungal, and other infection-causing microbes) counts on the skin of the patient just prior to the invasive procedure (e.g., surgery), reducing risk of post-operative or other infection in the patient. The kits and methods advantageously provide a convenient, relatively simple mechanism for such microbial counts to be reduced, and can be employed by the patient, e.g., in the days and hours just before the scheduled invasive procedure.

According to another embodiment, the kit and associated method may specifically and advantageously provide for two cleansings of the patient's skin, at spaced apart intervals. For example, the patient may cleanse the skin near the scheduled time of the invasive procedure (e.g., the morning of the procedure, or within about 2 hours of the procedure), while also having previously performed the same cleansing procedure several hours or a day or two earlier (e.g., the night before the procedure). Such a method may comprise providing a sealed kit including two subkits which may be identically configured, which are initially attached to one another, and which may be separated from one another to easily allow the user to perform the desired two treatments.

For example, such a method may comprise providing a sealed kit including two or more dry substrates, a first container and a second container, each including an antiseptic composition, a first tray and a second tray, wherein each tray has one or more recessed portions for holding the dry substrates and the antiseptic composition. The kit includes a flexible cover in contact with a top rim of the first tray to seal the kit, and a separable region (e.g., perforations) between the first tray and the second tray. The contents of each tray may be identical. The method further comprises separating the first tray from the second tray along the separable region such that the flexible cover is also separated into a first cover over the first tray, and a second cover over the second tray, each tray being independently sealed by its respective cover. The first cover may be removed from the first tray, one or more of the dry substrates contained therein may be placed within the recessed portion of the first tray, and the

(s) in the recessed portion may be dosed with the antiseptic composition. The dosed substrate may then be used to apply the antiseptic composition to an area of the skin proximate a location of the invasive procedure. This first cleansing may be done within 48 hours or 24 hours prior to the invasive procedure, but before a second cleansing (e.g., the night before the scheduled invasive procedure).

Later (e.g., the morning of the scheduled invasive procedure), the second cover may be removed from the second tray, one or more dry substrates may be placed in the recessed area of the second tray, the dry substrates may be dosed with the antiseptic composition, and the dosed substrate(s) used to apply the antiseptic composition to an area of the skin (e.g., the same area as cleansed with the first cleansing) proximate a location of the invasive procedure. This second cleansing may be done not more than 6 hours, not more than 4 hours, or not more than 2 hours prior to the invasive procedure.

According to an embodiment, a kit of the present invention includes one or more dry substrates, one or more containers of an antiseptic composition (e.g., a chlorhexidine gluconate antiseptic composition), one or more trays containing the one or more dry substrates and the container(s) of antiseptic composition, and a flexible cover extending over a top of the one or more trays to create a sealed kit.

Another embodiment of a kit according to the present invention may include two or more dry substrates, two or more containers of antiseptic composition, a first tray and a second tray, each containing one or more of the dry substrates and a container of antiseptic composition, a flexible cover extending over a top of the first and second trays, in contact with a top rim of the first tray and a top rim of the second tray to create a sealed kit, where the contents within each tray are sealed, and a separable region positioned between the first tray and the second tray to enable separation of the first tray from the second tray without opening the sealed kit (i.e., without opening the cover of either tray). In other words, such a kit may include two subkits, each subkit independently including contents needed for a cleansing treatment.

Further features and advantages of the present invention will become apparent to those of ordinary skill in the art in view of the detailed description of preferred embodiments below.

### BRIEF DESCRIPTION OF THE DRAWINGS

To further clarify the above and other advantages and features of the present invention, a more particular description of the invention will be rendered by reference to specific embodiments thereof which are illustrated in the drawings located in the specification. It is appreciated that these drawings depict only typical embodiments of the invention and are therefore not to be considered limiting of its scope. The invention will be described and explained with additional specificity and detail through the use of the accompanying drawings in which:

FIG. 1 is a perspective view of an exemplary dual tray kit according to an embodiment of the present invention;

FIG. 2 is a perspective view of an exemplary single tray kit otherwise similar to that of FIG. 1;

FIG. 3 shows a container of antiseptic composition locked within the corresponding recess of an exemplary tray of a kit;

FIG. 4 shows a cross-section through an exemplary tray including a textured portion in its bottom wall to maintain a dosed substrate above excess dosing antiseptic composition;

FIG. 5 shows how the two sections of an exemplary tray may serve as a drip tray so that excess antiseptic composition may be removed from the recessed portion where the substrate(s) are dosed;

FIG. 6 shows separation of two subkits of an exemplary dual tray kit (e.g., such as that of FIG. 1) from one another;

FIG. 7 shows removal of the flexible cover from a first tray;

FIG. 8 shows dosing of one or more substrates with the antiseptic composition after the cover is opened;

FIG. 9 shows a patient using the dosed substrate to wash their body prior to an invasive procedure;

FIG. 10 is a perspective view of an ornamental design for a tray packaged disinfectant delivery system;

FIG. 11 is a top view of the tray packaged disinfectant delivery system of FIG. 10;

FIG. 12 is a bottom view of the tray packaged disinfectant delivery system of FIG. 10;

FIG. 13 is a front end view of the tray packaged disinfectant delivery system of FIG. 10;

FIG. 14 is a rear end view of the tray packaged disinfectant delivery system of FIG. 10;

FIG. 15 is a first side view of the tray packaged disinfectant delivery system of FIG. 10; and

FIG. 16 is a second, opposite side view of the tray packaged disinfectant delivery system of FIG. 10.

### DETAILED DESCRIPTION

### I. Definitions

Before describing the present invention in detail, it is to be understood that this invention is not limited to particularly exemplified systems or process parameters that may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

The term “comprising” which is synonymous with “including,” “containing,” or “characterized by,” is inclusive or open-ended and does not exclude additional, unrecited elements or method steps.

The term “consisting essentially of” limits the scope of a claim to the specified materials or steps “and those that do not materially affect the basic and novel characteristic(s)” of the claimed invention.

The term “consisting of” as used herein, excludes any element, step, or ingredient not specified in the claim.

It must be noted that, as used in this specification and the appended claims, the singular forms “a,” “an” and “the” include plural referents unless the content clearly dictates otherwise. Thus, for example, reference to a “surfactant” includes one, two or more surfactants.

Various aspects of the present devices and systems may be illustrated by describing components that are coupled, attached, and/or joined together. As used herein, the terms “coupled”, “attached”, and/or “joined” are used to indicate either a direct connection between two components or, where appropriate, an indirect connection to one another through intervening or intermediate components. In contrast, when a component is referred to as being “directly coupled”, “directly attached”, and/or “directly joined” to another component, there are no intervening elements present.

Various aspects of the present devices, systems, and methods may be illustrated with reference to one or more exemplary embodiments. As used herein, the term “exemplary” means “serving as an example, instance, or illustration,” and should not necessarily be construed as preferred or advantageous over other embodiments disclosed herein.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In the application, effective amounts are generally those amounts listed as the ranges or levels of ingredients in the descriptions, which follow hereto. Unless otherwise stated, amounts listed in percentages (“wt %'s”) are in wt % (based on 100 weight % active) of the particular material present in the referenced composition, any remaining percentage typically being water or an aqueous carrier sufficient to account for 100% of the composition, unless otherwise noted. For very low weight percentages, the term “ppm” corresponding to parts per million on a weight/weight basis may be used, noting that 1.0 wt % corresponds to 10,000 ppm.

### II. Introduction

The present disclosure is directed to methods for reducing microbial counts on a patient's skin prior to an invasive procedure, as well as related kits employed in such methods. Such a kit may include one or more dry substrates, (e.g., cloths, non-woven towels, woven materials, sponges, etc.), a container containing an antiseptic composition, a tray with one or more recessed portions for holding the dry substrate(s) and the antiseptic composition, and a cover in contact with a top rim of the tray to seal the kit.

An exemplary method may include providing such a sealed kit, removing the cover from the tray, dosing one or more of the dry substrates with the antiseptic composition, and using the dosed substrate(s) to apply the antiseptic composition to at least one area of skin proximate a location of an invasive procedure to be performed.

### III. Exemplary Kits and Methods

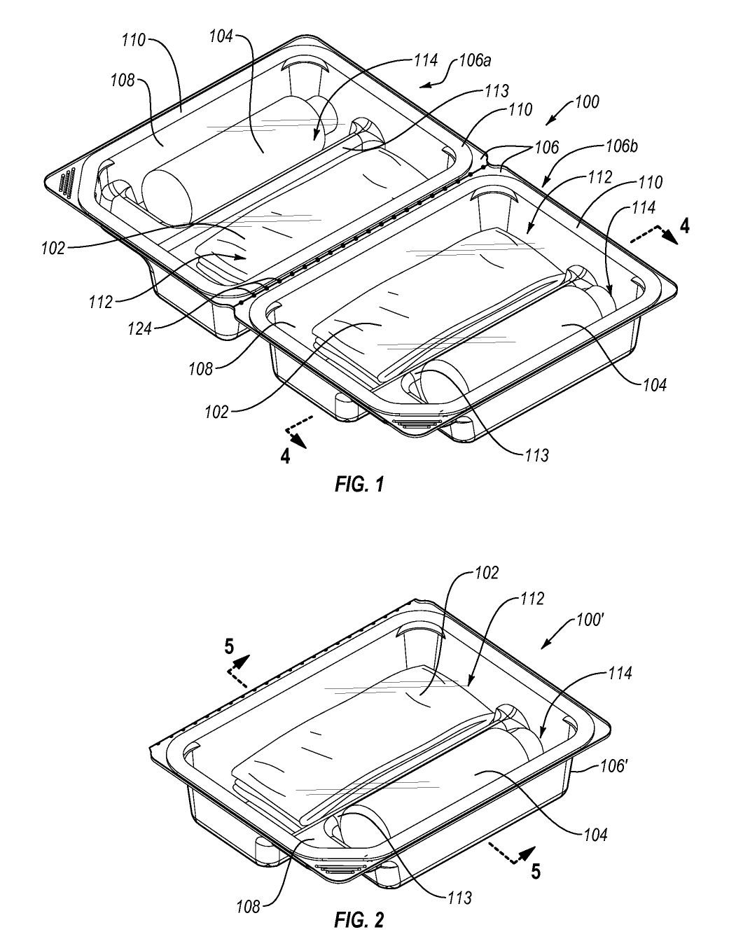

FIG. 1 shows an exemplary sealed dual tray kit 100. Kit 100 may include one or more initially dry substrates 102, a container (e.g., a bottle 104) of an antiseptic composition 105, and a tray 106 with one or more recessed portions for holding substrates 102 and container 104. A cover 108 may be provided in sealing contact with a top rim 110 of tray 106 to seal the contents (e.g., substrates 102 and container 104 of antiseptic composition 105) of the kit 100 within the tray 106. The contents sealed internally within tray 106, including the internal surfaces of tray 106 may advantageously have been sterilized after sealing to ensure a desired degree of sterility to the kit contents.

Kit 100 shown in FIG. 1 is a dual tray kit, effectively including two subkits 100a and 100b. As such, tray 106 includes two individual trays 106a and 106b, each of which may include identical contents (e.g., dry substrate(s) 102, container 104 of antiseptic composition, etc.), providing two single use subkits 100a, 100b within kit 100. FIG. 2 illustrates a single kit 100′, which includes a tray 106′, including contents similar to individual trays 106a or 106b of FIG. 1 (e.g., dry substrate(s) 102 and container 104 of antiseptic composition), but single kit 100′ may provide sufficient materials for only a single use. As such, in an embodiment, the provided kit may include only a single tray, rather than the dual tray shown in FIG. 1. Similarly, FIG. 2 may represent either individual tray 106a or 106b after trays 106a and 106b are separated from one another (e.g., just prior to use), or a kit that is provided with materials for just a single use. In any case, the tray 106′, 106a, or 106b provides one or more initially dry substrates 102 and container 104 of antiseptic composition 105 sealed within the corresponding tray.

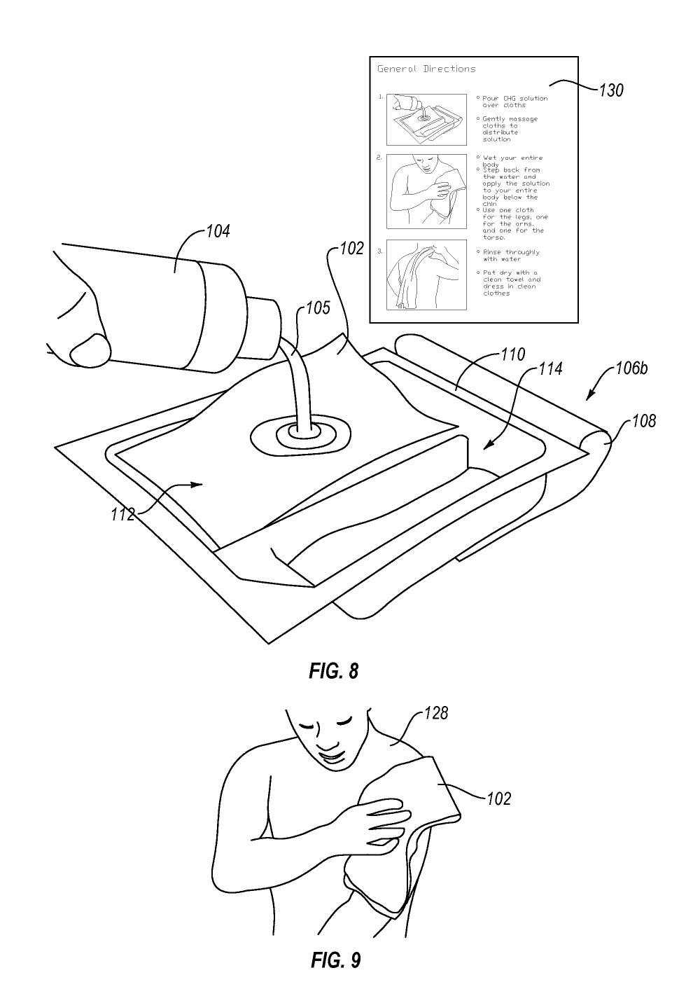

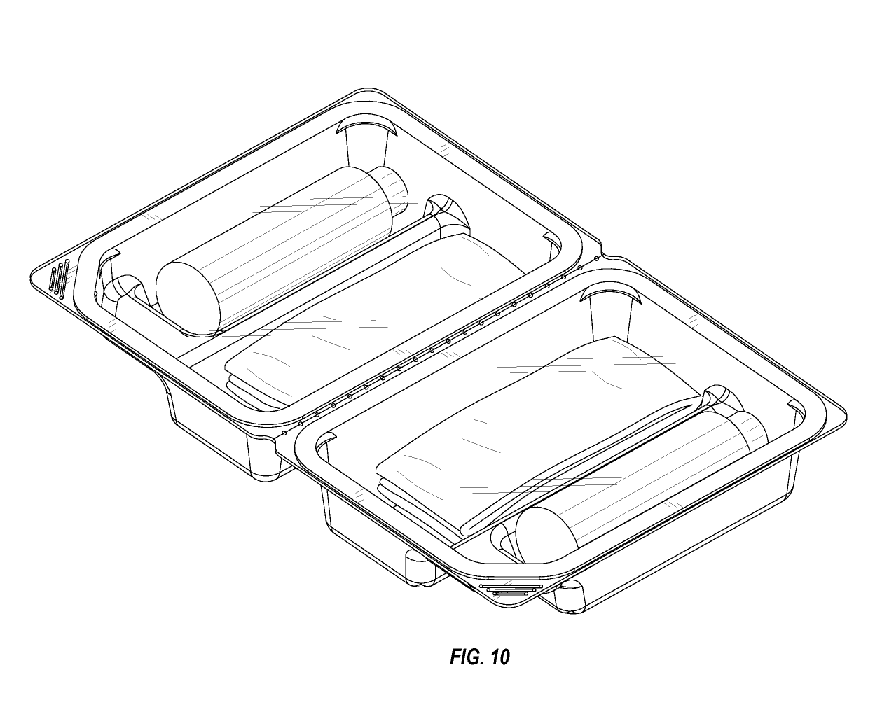

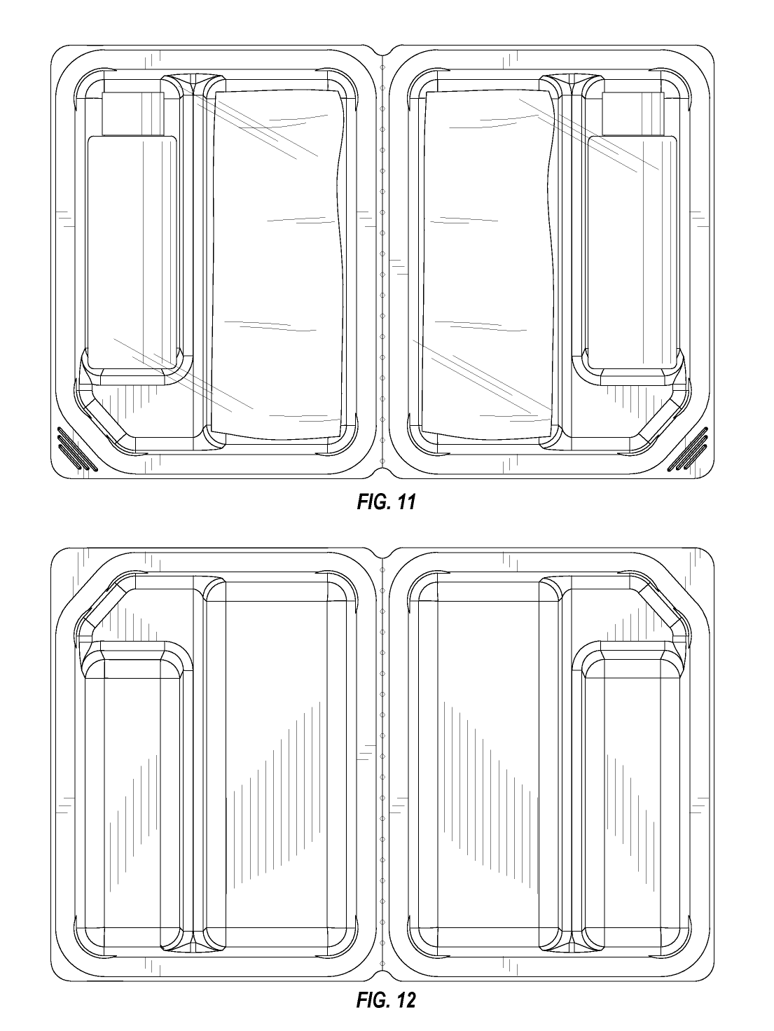

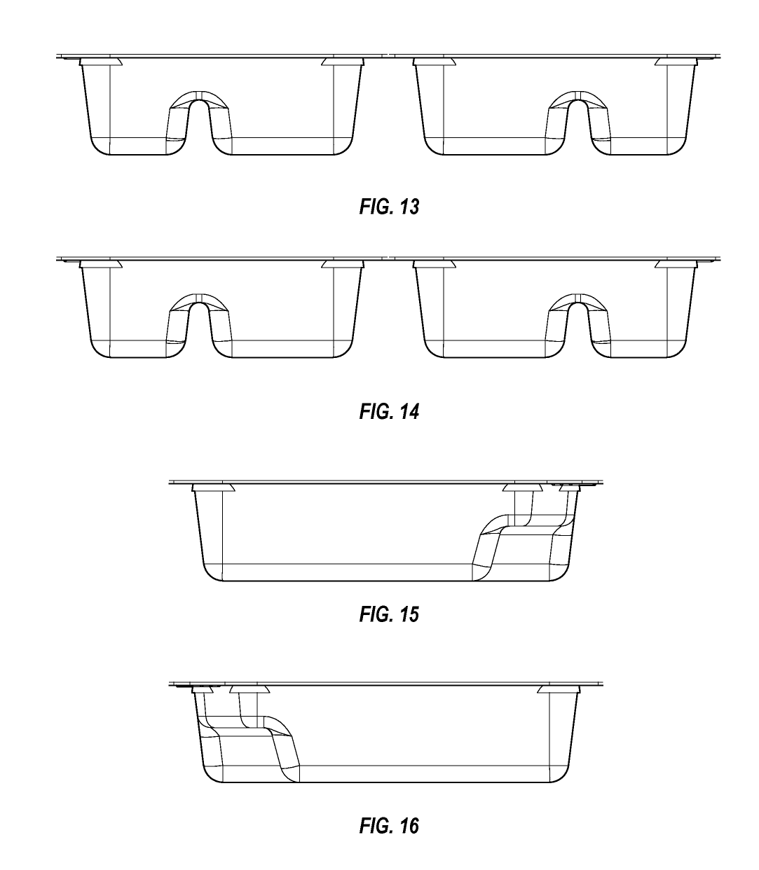

Although not shown in FIGS. 1 and 2 in order to better illustrate the other kit contents, kit 100′ of FIG. 2 or each subkit 100a and 100b of FIG. 1 may include instructions for use, e.g., printed on a waterproof (e.g., laminated) card which can advantageously be taken into the shower by the user, for reference during use, as desired. Such a card may be packed inside the kit (e.g., below the cover, between the cover and the container 104 and substrate(s) 102 held by the tray). Such an instruction card 130 is shown in conjunction with FIG. 8, showing an opened subkit.

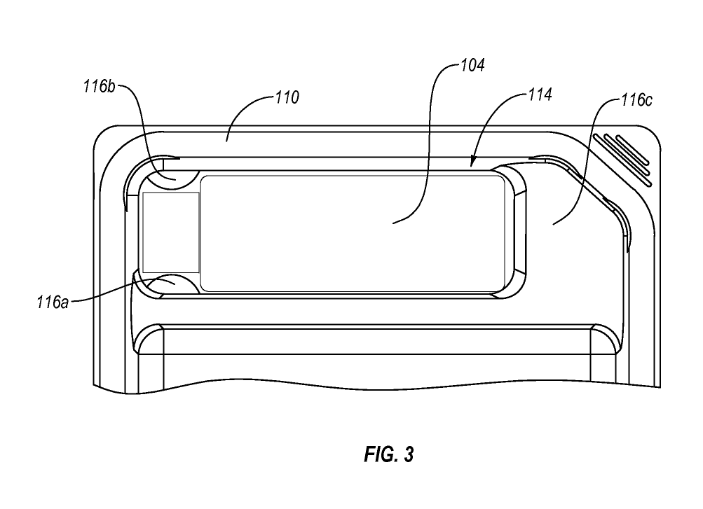

Trays 106′, 106a, and 106b each are illustrated as including two recessed portions 112 and 114. Recessed portions 112 and 114 may be separated by a divider 113, protruding inwardly towards cover 108, dividing the tray into two basins. Recessed portion 112 is configured to hold one or more dry substrates 102, while recessed portion 114 is configured to hold container 104. As shown in FIG. 3, recessed portion 114 may include one or more indentations extending into the volume defined by the recessed portion 114, to aid in orienting and/or retaining container 104 within portion 114. For example, FIG. 3 shows recessed portion 114 including two opposed indentations 116a and 116b extending from the sidewall defining recessed portion 114, which aid in orienting and securing or “locking” container 104 in place. Such indentations 116a and 116b may sandwich the lid or another portion of container 104 therebetween. The bottom of container 104 may rest against another indentation 116c extending from the sidewall defining recessed portion 114. Such a series of indentations 116 may aid in securing and orienting container 104 within recessed portion 114, holding container 104 in place as kit 100 or 100′ is moved, carried, transported, etc.

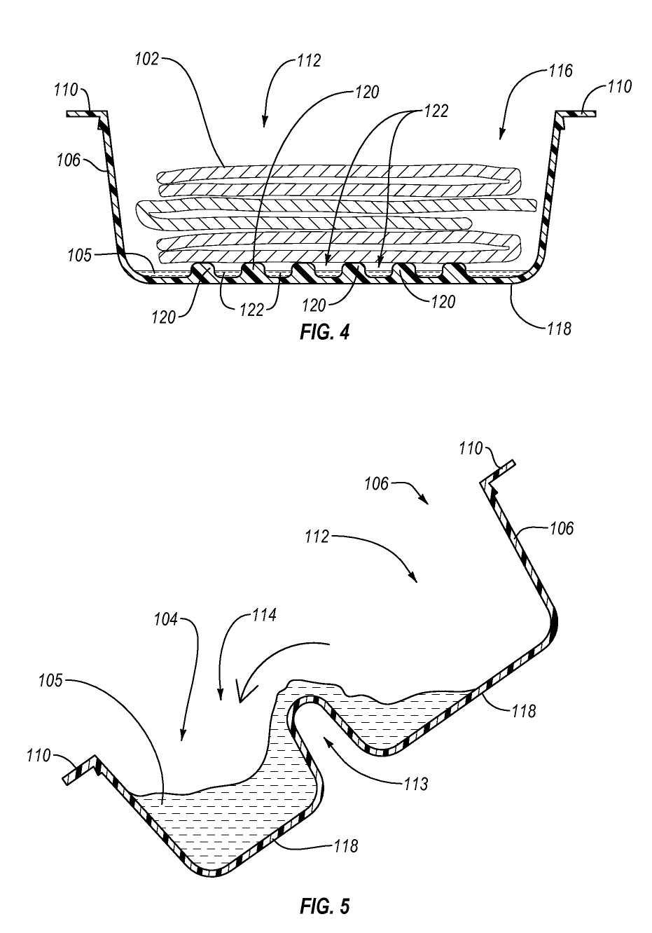

FIG. 4 illustrates a cross-sectional view through tray portion 112, which may be configured as a basin for holding dry substrate(s) 102. As illustrated, the bottom wall 118 of tray portion 112 may include a non-flat, textured surface thereon to raise substrate(s) 102 up above any excess antiseptic composition 105 as substrate(s) 102 are dosed with the antiseptic composition 105 initially stored within container 104. For example, as antiseptic composition 105 is poured or otherwise dispensed onto substrate(s) 102, at least some of composition 105 is absorbed within substrate(s) 102, while any excess may seep therethrough, collecting in recesses 122 between protruding portions 120 of the textured surface in bottom wall 118. In an embodiment, the textured surface may be provided by shapes, patterns, embossing logos or other branding within bottom wall 118, creating the desired non-flat, textured surface.

Substrate(s) 102 may comprise any suitable material. For example, such substrates may be formed of cloth, a non-woven material, polyurethane sponge, or any combinations thereof. Spunlaced non-woven materials are an example of suitable substrates. Spunlace processes typically employ high pressure water jets to entangle fibers in a manner to provide integrity and durability to the resulting fabric substrate. The substrates may be soft on skin, making it easy to apply the antiseptic composition. In any case, the substrate is capable of soaking up and holding a sufficient quantity of the antiseptic composition so as to allow a user to dose the substrate(s) with the composition, and then use the substrate(s) to apply the composition to the user's skin. Such substrate(s) may advantageously have sufficient structural integrity to not disintegrate during use. After a single use, the substrate(s) are typically discarded. An exemplary subkit for a single use may include a plurality of substrates (e.g., 3 substrates).

Tray 106 (e.g., which may comprise a single tray or dual trays 106a, 106b) may be thermoformed from a suitable plastic material. In one embodiment, the plastic material may be clear. The tray material may be translucent or transparent so that the patient or practitioner using the kit will be able to see the components of the kit. In another embodiment of the invention, the tray may be colored or opaque to protect the components of the kit that may be sensitive to light (e.g. degrading more quickly or less stable upon exposure to light). Examples of suitable plastics include various polymeric materials (e.g., polyolefins) that may be thermoformed, including, but not limited to polypropylene, polyethylene (e.g., high density polyethylene, low density polyethylene), ethylene/propylene copolymers, ethylene/butylene copolymers, vinyl and vinyl polymers, acrylic polymers, polyesters (e.g., polyethylene terephthalate (PET), polybutylene terephthalate), polyvinyl alcohol, polyamides, polyvinylchloride, polyvinylidene chloride, ethylene vinyl alcohol, polycarbonate, and any combinations or mixtures thereof Laminate materials including layers of different plastic materials may be employed. Various adjuvants may be included with the plastic material from which the tray is formed. For example, in an embodiment, inorganic oxides (e.g., metal oxides) may be incorporated to adjust (e.g., decrease) permeability of the resulting material to ethylene oxide penetration, where the kit is sterilized by exposure to ethylene oxide. Exemplary inorganic oxides include, but are not limited to silicon oxide, aluminum oxide, zinc oxide, magnesium oxide, and combinations thereof.

The tray may advantageously be rigid or semi-rigid. For example, it may have a rigidity that is greater than that of the flexible cover 108 that seals over the top rim of the tray, providing a basin (e.g., recessed portion 112) within which the substrates may be dosed with the antiseptic composition. The flexible cover 108 may be formed of a similar plastic material, but may be sufficiently thin so as to be flexible as compared to the rigid or semi-rigid tray. The flexible cover may be translucent or transparent so that the patient or practitioner using the kit will be able to see the components of the kit. In another embodiment of the invention, the flexible cover may be colored, metallic or opaque to protect the components of the kit that may be sensitive to light (e.g. degrading more quickly or less stable upon exposure to light). Alternatively, the opaque, colored or metallic flexible film may be used as an indication for the size or purpose/function of the tray to the patient or practitioner using the kit prior to a particular surgical procedure. It may be desirable to color code the kits to indicate which kit or should be used in preparation for a given surgical procedure or the color may indicate the type of actives in the antiseptic composition or the color may indicate where the kit may be used on a patient's skin/body.

The flexible cover may be a monolayer, multilayer or laminated material which is flexible and heat sealable to the tray. The cover 108 and/or tray 106 may comprise multiple layers laminated together, so as to provide desired barrier properties (e.g., desired degree of water impermeability, desired degree of air impermeability, desired degree of oxygen impermeability, etc.). In one embodiment, the semi-rigid tray may be substantially impermeable to gases used for sterilization (e.g. ethylene oxide, ozone, etc.) and at least a portion of the cover is sufficiently permeable to the sterilization gas so that the interior surfaces of the kit and its components are sterile before use.

The trays may be of any desired dimensions. In an embodiment, the depth of the basin defined by the tray of each subkit may be about 1.25 inch to about 2 inches. The length and width of an exemplary subkit may be about 8-8.5 inches, and about 5.5-6.5 inches, respectively. As shown in FIG. 1, the two subkits may initially be joined together, length to length.

FIG. 5 illustrates a cross-section through a single tray 106, showing both portions 112 and 114, separated by divider 113. As shown, divider 113 may comprise an inwardly oriented protrusion of bottom wall 118. Such a divider may allow one of the recessed portions to act as a drip tray, as composition 105 within one portion is caused to flow over divider 113, e.g., by tipping tray 106 (e.g., about 30°), as shown in FIG. 5, if desired. Such a configuration may be employed to prevent over-saturation of substrate(s) 102, even if the bottom wall 118 of basin 112 is not textured (e.g., the excess antiseptic composition may be poured out of basin 112).

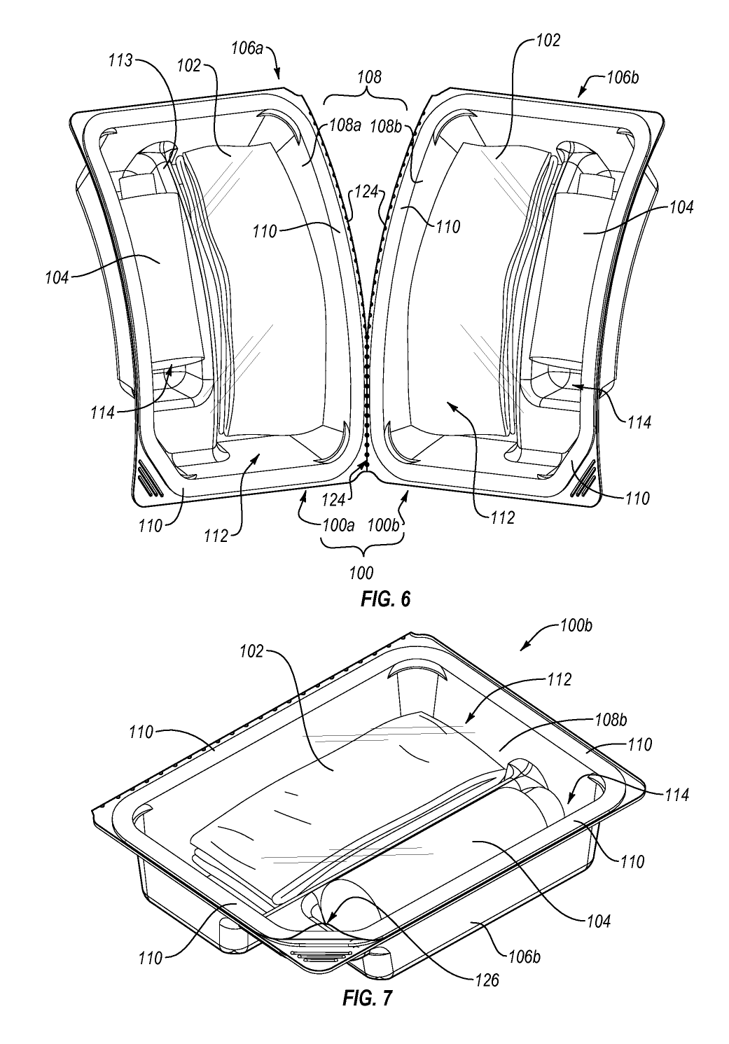

FIGS. 6-9 illustrate how the tray kits may be employed in an exemplary method. As described above, the kits may be provided so as to include a single tray, or in a dual tray configuration, as shown in FIGS. 2 and 1, respectively. A single tray kit such as tray kit 100′ shown in FIG. 2 may be provided and employed where a single application of antiseptic composition to a user's skin is desired. A dual tray kit, such as that shown in FIG. 1 advantageously provides sufficient materials for two separate applications, where the materials for each application are independently sealed within their own subkit (e.g., trays 106a and 106b) of a dual tray kit 100. Of course, two single tray kits could be used to provide two separate applications, as well.

As shown in FIG. 6, the individual trays 106a and 106b of kit 100 may be separated from one another, e.g., along a perforated area or other separable region disposed between the individual subkits, through flexible cover 108 and the underlying plastic initially joining trays 106a and 106b together. Any suitable perforation, die cutting, rouletting, or other structure providing for the adjacent trays to initially be joined together, and to be easily torn or otherwise separated from one another may be employed. The term perforation is used broadly, and includes perforations, die cutting, rouletting, and other structures resulting in a separable region providing discontinuous connection (e.g., alternating connected regions separated by intervening slits, holes, etc.) between the adjacent trays. FIG. 6 illustrates an example of such discontinuous connection provided by perforations 124, which facilitate tearing the two individual trays 106a and 106b apart.

Advantageously, cover 108 may be sealed against top rim 110 of each of trays 106a and 106b, on either side of the separable region (e.g., perforations 124), so that upon separation, each tray includes its own cover 108a, 108b which independently provides sealing of the respective tray contents. As such, a user is able to tear one subkit from the dual tray kit 100, and open one of the subkits (e.g., 100a or 100b) by removing cover 108a or 108b from the corresponding tray without having to open the other subkit. This advantageously allows the second subkit to remain sealed and sterile until its use, which may typically be hours or even a day or two after the first subkit is used.

FIG. 7 illustrates one of the subkits (e.g., first subkit 100b) alone. The user may open the subkit 100b by removing flexible cover 108b, e.g., by peeling corner 126 away from top rim 110. As shown in FIG. 7, a corner of flexible cover 108b may be provided with a free corner (e.g., 126) that is unsealed relative to underlying tray 106b so as to be easily grasped by the user, and peeled away from top rim 110, unsealing the contents of tray 106b. As shown, each top rim 110 may extend around the entire perimeter of tray 106b, and cover 108b may initially be sealed along the entire perimeter of top rim 110, providing an air-tight, water-tight seal therebetween. The free corner 126 of flexible cover 108b may extend beyond top rim 110.

As seen in FIG. 8, with cover 108b at least partially removed, instruction card 130 may be removed, container 104 initially disposed within recessed portion 114 may be removed, and the antiseptic composition 105 contained therein may be poured or otherwise dispensed onto the one or more dry substrate(s) 102 so as to dose the initially dry substrate(s) with the antiseptic composition. For example, container 104 may include a flip top cap to allow the composition 105 to be dispensed therethrough. This dosing may be performed with the substrate(s) 102 still within recessed portion 112 of tray 106b. Dosing substrate(s) 102 within recessed portion 112 provides an easy mechanism for dosing, soaking or otherwise impregnating substrate(s) 102 with composition 105, with minimal mess, as any excess composition 105 may simply collect within the bottom of recessed portion 112 (e.g., separated from impregnated substrate(s) 102 in recesses, as shown in FIG. 3). Substrate(s) 102 may be gently massaged (e.g., within or above recessed portion 112) to distribute the antiseptic composition into the substrate(s). As shown in FIG. 9, the dosed substrate 102 may then be used by the patient 128 to apply antiseptic composition 105 held therein to an area of the skin proximate a location of the invasive procedure to be performed.

This may be easily achieved in the shower or bathtub by the user as he or she is otherwise washing his or her body. For example, the user may use any shampoos, conditioners, soaps, or other cleansers as normally used, before applying the antiseptic composition. The user may wash his or her hands with soap and water prior to removing the cover from the tray. Once any cleansers such as those mentioned above have been used and rinsed away, the user may wet his or her body prior to application of the antiseptic composition, and step back from any shower spray while applying the composition using the dosed substrate 102. Cleansing with the antiseptic composition 105 may be similar to using liquid soap, as the dosed substrate 102 is used to scrub the skin. Once the antiseptic composition has been applied, the area of skin where the antiseptic composition is applied may be rinsed with water alone (e.g., no soap, etc.). Before rinsing, the user may pause in order to allow the antiseptic composition to settle on the skin (e.g., about 10 seconds to about 3 minutes). After such rinsing with water, the treated area of skin may then be dried. In an embodiment, drying may achieved by patting the area of skin with a clean towel, or by air drying the area of skin where the antiseptic composition was applied. Application of lotions, deodorants, hair styling products, or other similar formulations may be avoided after treatment, as they may interfere with the bacteria killing effect of the antiseptic composition.

The antiseptic composition may be applied to any or all areas of the skin where its use is not contraindicated (e.g., applied to the entire body below the chin, so as to avoid eyes, ears, and mouth). Application to the genitals may also be avoided. A plurality of substrates 102 (e.g., 3 substrates) may be provided within a single subkit 100a. For example, the user may use one substrate for the legs, one for the arms, and one for the torso. The substrates may be of any desired size. In an embodiment, each substrate may measure about 10×13 inches when unfolded. The plurality of substrates may be dosed with a single container (e.g., 4 fl. oz.) of the antiseptic composition.

Each subkit (e.g., 100a, 100b) may include instructions for use, e.g., printed on a waterproof (e.g., laminated) card which can be taken into the shower by the user, for reference during use, as desired. FIG. 8 illustrates such a card 130. The instruction card may include space for additional written instructions from the patient's doctor (e.g., the doctor or other practitioner may highlight those areas of the body to be treated with the antiseptic composition).

Kit 100 shown in FIG. 1 advantageously includes two subkits 100a and 100b, which can be separated from one another, with each subkit still remaining sealed. Unsealing or opening of one subkit advantageously does not unseal the second subkit. Such a kit allows a user to apply the antiseptic composition twice, at spaced apart intervals prior to the invasive procedure. For example, a user may use one subkit the night before the invasive procedure, and use the other subkit the morning of the procedure. Such a two stage application of the antiseptic composition further reduces the microbial count on the patient's skin as compared to a single application, which may further reduce any risk of infection.

In an embodiment, one subkit may be used to apply the antiseptic composition within 48 hours, or within 24 hours prior to the invasive procedure (e.g., the night before). The second subkit may be used to repeat application of the antiseptic composition not more than 8 hours, not more than 6 hours, not more than about 4 hours, or not more than about 2 hours prior to the invasive procedure (e.g., the morning of the procedure). In an embodiment, the time between use of the first subkit and use of the second subkit may be from about 2 hours to 48 hours, from about 4 hours to about 24 hours, or from about 4 hours to about 12 hours. For example, in an embodiment, the first application may be at least about 4 hours (e.g., the night before) prior to the invasive procedure, and the second application may be within about 2 hours prior to the procedure (e.g., the morning of the procedure). More than two applications (e.g., 3), and kits including more than 2 subkits may also be provided.

Of course, the kits and subkits may be employed by a medical practitioner to treat a patient's skin in-facility (e.g., in the hospital, etc.), rather than having the patient self-treat at home.

The antiseptic composition stored within the container of the kit may include an antiseptic agent therein. Examples of such antiseptics include, but are not limited to chlorhexidine, or a chlorhexidine salt, such as chlorhexidine gluconate (CHG). Other antiseptics (e.g., povidone iodine (PVP-I), iodine, or other iodine complexes may also be suitable for use. An alcohol (e.g., C₁-C₄ alcohols) may also be included (e.g., methanol, ethanol, isopropyl alcohol, butyl alcohol, combinations thereof, etc.). The composition may be an aqueous solution.

In an embodiment, the antiseptic composition stored within the container of the kit may be a chlorhexidine gluconate (CHG) composition. For example, the antiseptic composition may be an aqueous solution including from about 2% to about 4% of CHG, and from about 1% to about 45% of an alcohol (e.g., one or more alcohols). The majority of the balance of the composition may be water. Other additives may optionally be included, such as, but not limited to, colorants, dyes, surfactants, pH adjusters, thickeners, fragrances, and mixtures or combinations thereof. Where a colorant or dye is present, the person applying the antiseptic composition may use the dye as an indicator of where the composition has been applied to ensure proper coverage of the patient's skin. If washing with water is indicated after the antiseptic composition has been applied, the dye or colorant may also serve as a reminder or visual cue that the antiseptic composition is persistent after the rinsing and remind the patient or healthcare professional administering the solution not to use soaps or dry the treated area too vigorously.

The antiseptic composition may be manufactured within a controlled environment to ensure it is also aseptic, meaning it is generally sterile (e.g., free of disease causing microbes), where contaminants are controlled. The kit may also be sterilized once manufactured. As will be apparent from the above, in addition to being aseptic, the composition is also antiseptic, where an antiseptic agent is included therein, so as to allow its application to skin so as to kill microbes, reducing the microbial count on a patient's skin when applied prior to an invasive procedure, so as to reduce any possibility of post-surgery infection. Thus, the composition may be aseptic, and also antiseptic.

The sealed kits may be sterilized, as desired. Sterilization may be by any suitable technique, including, but not limited to, exposure to gaseous ethylene oxide, dry heat sterilization, steam sterilization, exposure to ozone, exposure to radiation (e.g., gamma radiation, UV radiation), combinations thereof, etc. Sterilization may be performed after the kit contents are sealed within the tray. If desired, the container 104 and its antiseptic composition 105 may be sterilized separately (e.g., it may be sterilized itself, prior to placement within the tray, after which the kit as a whole may be sterilized). As a result of such sterilization, the interior surfaces of the tray, as well as the contents of the tray may be sterile.

Any ethylene oxide sterilization may be such so as to ensure that less than 250 ppm, less than 100 ppm, or less than 25 ppm of residual ethylene oxide is present within the antiseptic composition. In an embodiment, one or more of the body of container 104, the tray, or the flexible cover may each independently exhibit permeability to gaseous ethylene oxide of 20 mg/hr/cm² or less.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A method of reducing microbial count on a patient's skin prior to an invasive procedure, the method comprising:
(A) providing a sealed kit comprising:
one or more dry substrates;
(ii) a container containing an antiseptic composition;
(iii) a tray with one or more recessed portions for holding the dry substrates and the antiseptic composition; and
(iii) a cover in sealing contact with a top rim of the tray to seal the kit;
(B) removing the cover from the tray;
(C) dosing the dry substrate with the antiseptic composition within one of the one or more recessed portions of the tray; and
(D) using the dosed substrate to apply the antiseptic composition to at least one area of skin proximate a location of the invasive procedure to be performed.

2. The method of claim 1 further comprising: (E) rinsing the area of skin with water alone after (D).

3. The method of claim 2 further comprising: (F) drying the area of skin after (E).

4. The method of claim 3 wherein drying the area of skin is done by patting the area of skin with a clean towel.

5. The method of claim 3 wherein drying the area of skin is done by air drying the area of skin.

6. The method of claim 1 further comprising: having the patient wash his or her hands with soap and water prior to (B).

7. The method of claim 1 wherein the antiseptic composition is applied over all areas of the skin where the antiseptic composition is not contraindicated.

8. The method of claim 1 wherein the antiseptic composition comprises:
(A) about 2% to 4% by weight of chlorhexidine gluconate;
(B) about 1% to 45% by weight of an alcohol; and
(C) optionally, an additive selected from the group consisting of colorants, dyes, surfactants, pH adjusters, thickeners, fragrances, and any mixtures or combinations thereof.

9. The method of claim 1 wherein the tray is made of a thermoformed plastic material.

10. The method of claim 1 wherein the tray comprises a first recessed portion for holding the container of antiseptic composition.

11. The method of claim 10 wherein the tray comprises a second recessed portion for holding the one or more dry substrates.

12. The method of claim 11 wherein the second recessed portion has a textured surface in a bottom wall thereof.

13. The method of claim 1 wherein (A) through (D) are completed within 48 hours prior to the invasive procedure.

14. The method of claim 13 wherein (A) through (D) are repeated a second time within about 8 hours prior to the invasive procedure.

15. A method of reducing microbial count on a patient's skin prior to an invasive procedure, the method comprising:
(A) providing a sealed kit comprising:
(i) two or more dry substrates;
(ii) a first container and a second container, each containing an antiseptic composition;
(iii) a first tray and a second tray, wherein each tray has one or more recessed portions for holding the dry substrates and the antiseptic composition;
(iv) a flexible cover in sealing contact with a top rim of the first tray to seal the kit;
(v) a separable region between the first tray and the second tray;
(B) separating the first tray from the second tray along the separable region such that the flexible cover is also separated into a first cover over the first tray and a second cover over the second tray;
(C) removing the first cover from the first tray;
(D) placing one or more of the dry substrates with one of the one or more recessed portions of the first tray;
(E) dosing the one or more dry substrates with the antiseptic composition; and
(F) using the dosed substrate to apply the antiseptic composition to an area of skin proximate a location of the invasive procedure, within 48 hours prior to the invasive procedure;
(G) removing the second cover from the second tray;
(H) placing one or more of the dry substrates within one of the one or more recessed portions of the second tray;
(I) dosing the one or more dry substrates with the antiseptic composition; and
(J) using the dosed substrate to apply the antiseptic composition to an area of skin proximate a location of the invasive procedure, within about 8 hours prior to the invasive procedure.

16. The method of claim 15 wherein the one or more dry substrates are selected from the group consisting of: a nonwoven material, a polyurethane sponge, or any combinations thereof.

17. The method of claim 15 wherein the antiseptic composition comprises:
(A) chlorhexidine gluconate;
(B) an alcohol; and
(C) an additive selected from the group consisting of colorants, dyes, surfactants, pH adjusters, thickeners, fragrances, and any mixtures or combinations thereof.

18. The method of claim 15 wherein the first and second trays are made of a thermoformed plastic material.

19. The method of claim 15 wherein the first tray and the second tray each comprise a first recessed portion for holding the container of antiseptic composition.

20. The method of claim 19 wherein the first tray and the second tray each comprise a second recessed portion for holding the one or more dry substrates.

21. The method of claim 20 wherein each second recessed portion has a textured surface in a bottom wall thereof.

22. A kit for reducing microbial count on a patient's skin prior to an invasive procedure, the kit comprising:
(A) one or more dry substrates selected from the group consisting of: nonwoven susbtrates, woven substrates and sponges, and any combinations thereof
(B) one or more containers of chlorhexidine gluconate antiseptic composition;
(C) one or more trays containing the one or more dry substrates and the containers of antiseptic composition; and
(D) a flexible cover extending over a top rim of the one or more trays to create a sealed kit.

23. The kit of claim 22 wherein interior surfaces of the one or more trays sealed within the kit are sterile.

24. The kit of claim 22 wherein the chlorhexidine gluconate antiseptic composition further comprises:
(A) an alcohol; and
(B) an additive selected from the group consisting of colorants, dyes, surfactants, pH adjusters, thickeners, fragrances, and any mixtures or combinations thereof.

25. The kit of claim 22 wherein the one or more trays are made of a thermoformed plastic material.

26. The kit of claim 22 wherein the one or more trays comprise a first tray and a second tray, the first tray and the second tray each contains one or more of the dry substrates and a container of antiseptic composition.

27. The kit of claim 22 wherein the one or more trays each comprise a first recessed portion for holding a container of antiseptic composition.

28. The kit of claim 27 wherein the one or more trays each comprise a second recessed portion having a textured surface in a bottom wall thereof.

29. A kit for reducing microbial count on a patient's skin prior to an invasive procedure, the kit comprising:
(A) two or more dry substrates;
(B) two or more containers of chlorhexidine gluconate antiseptic composition;
(C) a first tray and a second tray each containing one or more of the dry substrates and a container of antiseptic composition;
(D) a flexible cover extending over a top of the first and second trays, in sealing contact with a top rim of the first tray and a top rim of the second tray to create a sealed kit; and
(E) a separable region positioned between the first tray and the second tray to enable separation of the first tray from the second tray without opening the sealed kit.

30. The kit of claim 29 wherein the first and second trays are made of a thermoformed plastic material.

31. The kit of claim 29 wherein the first and second trays each comprise a first recessed portion holding a container of antiseptic composition.

32. The kit of claim 29 wherein the first and second trays each comprise a second recessed portion having a textured surface in a bottom wall thereof.

33. The kit of claim 29 wherein the separable region comprises a perforated area through the flexible cover and plastic between the first and second trays which plastic initially joins the first and second trays together.
